(19) United States
(12) Patent Application Publication
(10) Pub. No.: US 2024/0173349 A1
(43) Pub. Date: May 30, 2024

(54) HEAD LICE TREATMENT

(71) Applicant: Nitoff Limited, London (GB)

(72) Inventors: Deirdre Gertrude Lillian Wright, London (GB); Joseph Cincotta, Trumbull, CT (US)

(73) Assignee: Nitoff Limited, London (GB)

(21) Appl. No.: 18/284,523

(22) PCT Filed: Feb. 10, 2023
(86) PCT No.: PCT/US2023/012842

§ 371 Date: Sep. 27, 2023

(30) Foreign Application Priority Data
Feb. 14, 2022 (GB) 2201942.6

(51) Int. Cl. A61K 31/80; A61K 9/00; A61K 47/10; A61P 33/14

(52) U.S. Cl. CPC A61K 31/80; A61K 9/0014; A61K 47/10; A61P 33/14

(57) ABSTRACT

A composition, for use in controlling lice or their ova on an animal subject, comprises a reactive copolymer formed from two or more monomers and having a hydrophobic conditioning portion comprising a siloxane group, a reactive crosslinking group comprising a silanol, a hydrophilic portion comprising one or more of an ethoxylate group and a propoxy late group and a surface linking portion comprising an amine or quaternary ammonium group, the copolymer forming in-situ crosslinks when dried on hair. A method of controlling lice or their ova on an animal subject comprises applying to the head of the animal subject such a composition.

### FIELD OF THE INVENTION

The invention is concerned with compositions and methods for the treatment of head lice on animals, including humans.

### BACKGROUND TO THE INVENTION

Head lice treatment according to the present invention is neither a treatment to kill head lice nor the use of a repellent to expel them. Instead, this invention results from a new approach to the protection of hair when it is under attack.

The hair is the food and hydration source for the egg of the louse. In order to directly protect the hair from the louse egg drawing sustenance from the keratin, a barrier is created between the hair and the egg, cutting off its essential nourishment. Consequently, the egg withers and dies, unable to come to fruition. If the eggs are unable to develop and hatch the infestation naturally ends.

This approach creates a very different situation for the louse to deal with. Its environment has changed. Its source of food is untainted, its ability to breed is not attacked and its ability to breathe is not threatened. No harm comes to it or the human who has traditionally had to apply lice treatments which include potentially harmful chemicals or pesticides. Instead, the hair environment in which the louse lives, hides and lays its eggs is altered to the extent that the louse's ability to bring its young to fruition is removed.

The invention results from the consideration of a hair treatment expert rather than from the entomological or naturalist perspectives that have previously been adopted. A textile technology ingredient, synthesised in a specific way, is applied to create a distinctive and safe barrier, with the added benefit of conditioning the hair without leaving any greasy deposit.

Additionally, this is not a barrier which the louse is likely to overcome through evolution because it concerns itself with securely altering the external environment rather than directly attacking the louse. This invention liberates the hair itself and cancels out its previous role in the relationship between the hair and the louse.

### STATEMENTS OF THE INVENTION

According to the present invention, there is provided a composition for use in controlling lice or their ova on an animal subject, the composition comprising a reactive copolymer formed from two or more monomers and having a hydrophobic conditioning portion comprising a siloxane group, a reactive crosslinking group comprising a silanol, a hydrophilic portion comprising one or more of an ethoxylate group and a propoxylate group and a surface linking portion comprising an amine or quaternary ammonium group, the copolymer forming in-situ crosslinks when dried on hair.

Applying such a composition to wet hair by means of a spray, lotion, shampoo, conditioner, hair treatment or hair styling product creates, when dried, a lightweight non greasy durable in-situ crosslinked hydrophobic network on the hair fibre's surface. The composition surrounds each hair strand and acts as a durable shield such that when lice lay their eggs on the treated hair surface they are prohibited from feeding off the keratin substrate and so do not mature within their gestation period but rather dry up and die.

Preferably, the surface linking portion has an affinity to keratinaceous substrates.

A preferred copolymer is one which may be represented by the following repeat structure:

-[—(Si(CH₃)₂—O)x-N(SiOR)—(CH₂CH₂—O)y-(CH₂—CH(CH₃)—O)z-NR—]-
in which the Rs are the same or different and represent organic radicals, x is at least 1 and y and z are 0 or above with at least one of y and z being at least 1. The portion —(Si(CH₃)₂—O)_{χ}— provides hydrophobicity, the portion —N(SiOR)— provides crosslinking durability, the portions (CH₂CH₂—O) and (CH₂—CH(CH₃)—O) provide ease of formulation and low build-up and the portion —NR— provides affinity to keratin.

Preferably, the composition comprises one or more of a low molecular weight cationic conditioning agent, a low molecular weight glycol, and a fluorinated silicone polymer.

Preferably, the proportion of the reactive copolymer in the composition is 0.1 to 5% by weight, more preferably from 0.5 to 3.0% by weight.

Preferably, the composition comprises one or more of glycerol, propylene glycol, a propanediol, a butanediols, dipropylene glycol, butylene glycol, 1,3-butane diol, pentylene glycol, hexylene glycol and caprylyl glycol.

Preferably, the composition comprises at least one long chain fatty alcohol having 10 to 22 carbon atoms, more preferably, 12 to 18 carbon atoms.

Preferably, the composition comprises one or more of the fatty alcohols lauryl alcohol, myristyl alcohol, cetyl alcohol, stearyl alcohol, eicosanyl alcohol and behenyl alcohol.

Preferably, the proportion of fatty alcohol in the composition is from 0.5 to 5% by weight, more preferably from 0.75 to 3.0% by weight.

The present invention further provides a method of controlling lice or their ova on an animal subject, the method comprising applying to the head or coat of the animal subject a composition of the invention.

Preferably, the composition is applied to wet hair by means of a spray, lotion, shampoo, conditioner, hair treatment or hair styling product and then dried.

The composition is preferably applied to wet hair by means of a leave in treatment.

### DETAILED DESCRIPTION OF THE INVENTION

The invention will now be described in more detail, by way of examples.

Research was undertaken to establish the effectiveness of a composition of use in the present invention. The method used in the research involved leave on protection for the hair that was aimed at preventing the head louse egg from feeding, developing and hatching.

The composition contains a linear amino silane modified silicone polyether copolymer, in particular, a material known as Silsoft CLX-E which is manufactured by Momentive Performance Materials Inc. It comprises Polysilicone-29 (an epoxy amino silane copolymer) and dipropylene glycol. The composition of Silsoft CLX-E is as follows:
Dipropylene glycol: 30-60% CAS #25265-71-8 Water: 30-60% CAS #7732-18-5
Polysilicone 29: 10-30% CAS #1040921-50-3
Polysilicone-29 is a linear amino silane modified silicone polyether copolymer having a molecular weight of 20,000-30,000 g/mole.
Silsoft CLX-E is soluble in ethanol, and propylene glycol. It is dispersible in water and insoluble in mineral oil, cyclopentasiloxane and isopropyl myristate

The non-reactive amine or quaternary ammonium group portion of Polysilicone 29 aligns itself to the hair shaft and improves adhesion to hair. The polyether portion assists water dispersibility as well as the ease of formulation and assists the hydrophilic/hydrophobic balance leading to self-limiting deposition.

The siloxane portion acts to form a flexible film on keratinaceous substrates. The reactive Silanol portion cross-links to prolong the adhesion through multiple washes.

### Research Methodology

Access tests were conducted using fresh live head lice to infest a volunteer subject head which was already clear of nits or head lice.

Volunteer subjects were across a range of hair types, the composition's effectiveness could be tested in different hair conditions.

In all 17 tests were conducted.

### Research Format

The test format was as follows:

### Day 1 Planting

Hair was washed with the sulphate free shampoo removing any deposits from the hair and dried
No other products were applied to the hair
5 or 6 adult female head lice were then inserted into the hair
Hair was worn back
The head lice were left to lay their eggs undisturbed

### Day 4 Extraction and Application of the Protective Product+Observation

Hair washed with a sulphate free shampoo and dried
Hair divided into 8 sections and forensically examined section by section recording the presence of the head lice and of any identifiable eggs
Lice extracted
Eggs identified
   Numbers counted and position marked on an 8-section grid of the head
   Description of each egg noted
      N.B. Because Head Lice Eggs Take 7 to 11 Days from being Laid to Hatching—the Entire Test is Run to Day 14 or 15

### Day 7 OR 8 Observation

Hair divided into 8 sections and forensically examined section by section recording the presence of any lice and all the eggs
Presence of any lice noted if found
Eggs identified
   Numbers counted and position marked on 8 section grid of the head
Description of each egg noted

### Day 11 Observation

Hair divided into 8 sections and forensically examined section by section recording the presence of any lice and of the eggs
Presence of any lice noted
Eggs identified
   Numbers counted and position marked on an 8-section grid of the head
Description of each egg noted

### Day 14 OR 15 Observation and Egg Extraction

Hair divided into 8 sections and forensically examined section by section recording the presence of the lice and of the eggs
Presence of any lice noted
Eggs identified
   Numbers counted and position marked on an 8-section grid of the head
Description of each egg noted
Finally, the eggs cleared out of the hair

### Results

Over the 11-day period when the protective product was in the hair, from the extraction of the planted adult head lice to the completion of the test, no eggs hatched.

Other suitable silane crosslinking agents which may be used in the present invention include the following reactive copolymers containing silanol groups:
products of Croda: CRODASONE™ cystine PE (cystine bis-PG-propyl silanetriol), CRODASONE™ W (hydrolyzed wheat protein PG-propyl silanetriol), CRODASONE™ P (hydrolyzed pea protein PG-propyl silanetriol), and KERAVIS™ PE (hydrolyzed vegetable protein PG-propyl silanetriol);
products of Momentive: SILQUEST™ A-187 silane; and
products of Evonik: DYNASYLAN® GLYEO (3-glycidyloxypropyltriethoxysilane) and DYNASYLAN® GLYMO (3-glycidyloxypropyl-trimethoxysilane).

While only several embodiments of the present invention have been described, it will obvious to those skilled in the art that many modifications may be made to the present invention without departing from the spirit and scope thereof.

What is claimed is:

1. A composition for use in controlling lice or their ova on an animal subject, the composition comprising a reactive copolymer formed from two or more monomers and having a hydrophobic conditioning portion comprising a siloxane group, a reactive crosslinking group comprising a silanol, a hydrophilic portion comprising one or more of an ethoxylate group and a propoxylate group and a surface linking portion comprising an amine or quaternary ammonium group, the copolymer forming in-situ crosslinks when dried on hair.

2. A composition according to claim 1, wherein the surface linking portion has an affinity to keratinaceous substrates.

3. A composition according to claim 1, wherein the composition comprises one or more of a low molecular weight cationic conditioning agent, a low molecular weight glycol, and a fluorinated silicone polymer.

4. A composition according to claim 1, wherein the proportion of the reactive copolymer in the composition is 0.1 to 5% by weight.

5. A composition according to claim 4, wherein the proportion of the reactive copolymer in the composition is 0.5 to 3.0% by weight.

6. A composition according to claim 1, wherein the composition comprises one or more of glycerol, propylene glycol, a propanediol, a butanediols, dipropylene glycol, butylene glycol, 1,3-butane diol, pentylene glycol, hexylene glycol and caprylyl glycol.

7. A composition according to claim 1, wherein the composition comprises at least one long chain fatty alcohol having 10 to 22 carbon atoms.

8. A composition according to claim 7, wherein the composition comprises at least one long chain fatty alcohol having 12 to 18 carbon atoms.

9. A composition according to claim 1, wherein the composition comprises at least one of the fatty alcohols lauryl alcohol, myristyl alcohol, cetyl alcohol, stearyl alcohol, eicosanyl alcohol and behenyl alcohol.

10. A composition according to claim 7, wherein the proportion of fatty alcohol in the composition is from 0.5 to 5% by weight.

11. A composition according to claim 10, wherein the proportion of fatty alcohol in the composition is from 0.75 to 3.0% by weight.

12. A method of controlling lice or their ova on an animal subject, the method comprising the step of applying to the head of the animal subject a non-toxic composition comprising a reactive copolymer formed from two or more monomers and having a hydrophobic conditioning portion comprising a siloxane group, a reactive crosslinking group comprising a silanol, a hydrophilic portion comprising one or more of an ethoxylate group and a propoxylate group and a surface linking portion comprising an amine or quaternary ammonium group, the copolymer forming in-situ crosslinks when dried on hair.

13. A method according to claim 12, wherein the composition is applied to wet hair via a spray, lotion, shampoo, conditioner, hair treatment or hair styling product, and then drying the hair.

14. A method according to claim 12, wherein the composition is applied to wet hair via a leave in treatment.
